(19) United States
(12) Patent Application Publication
(10) Pub. No.: US 2009/0171356 A1
(43) Pub. Date: Jul. 2, 2009

(54) Bone Repositioning Apparatus and System

(75) Inventor: Peter M. Klett, Horgen (CH)

Correspondence Address: VAN N. NGUY;IBM CORPORATION, ALMADEN RESEARCH CENTER, INTELLECTUAL PROPERTY LAW DEPT. C4TA/J2B, 650 HARRY ROAD, SAN JOSE, CA, 95120-6099, US

(73) Assignee: International Business Machines Corporation, Armonk, NY (US)

(21) Appl. No.: 11/968,585

(22) Filed: Jan. 2, 2008

(51) Int. Cl. A61B 17/00; A61B 17/58; A61F 2/48; A61F 5/00

(52) U.S. Cl. 606/54; 606/90; 623/24; 602/39; 602/2

(57) ABSTRACT

The present invention provides an apparatus and system for bone repositioning. The bone-repositioning apparatus includes an actuator controller configured to transmit a series of coordinated signals; an outer sleeve dimensioned to encircle a body limb, including fragments of a fractured bone within the body limb; and a plurality of individually operable actuators, each actuator connected to the actuator controller and configured to receive one or more of the coordinated signals, each actuator comprising a member configured to protract and retract into and out of an interior portion of the outer sleeve to exert a predetermined force on the body limb and on at least one of the bone fragments. A bone-repositioning system including the bone-repositioning apparatus is also described, including a computer program product that partially or fully automates the repositioning process depending on the application.

### BACKGROUND

1. Field of Invention

This invention relates to bone repositioning and, in particular, to an apparatus and system for bone repositioning.

2. Related Art

Repositioning a bone after a fracture is typically done manually by a trained highly individual, e.g., a medical doctor. To reposition the fractured bone within a body limb (e.g., a forearm or thigh), a doctor typically requests an image (e.g., an x-ray image) of the limb be taken, views the image to determine the fractured bone's position within the limb, repositions the bone manually based on the image, and then requests another image of the limb be taken to confirm that he or she has repositioned the bone correctly. In some cases, the doctor may use fluoroscopy to reposition the bone manually. This manual repositioning of the fractured bone requires significant experience on the part of the person repositioning the bone. Additionally, if the fractured bone is a large bone, e.g., a femur bone in a fully grown adult, the strength required to manually reposition the bone may be great. There are risks of inaccuracies and latent damage during the repositioning phase. There are further risks of inadvertently altering the position of the newly repositioned bone during subsequent phases, e.g., while placing an orthopedic cast on the limb to immobilize the limb so that the bone can heal.

Thus, what is needed is an improved apparatus and system for repositioning a bone.

### BRIEF SUMMARY

The invention provides a non-invasive bone-repositioning apparatus including an actuator controller configured to transmit a series of coordinated signals; a rigid outer sleeve dimensioned to encircle a body limb, including fragments of a fractured bone within the body limb, the outer sleeve having an exterior portion, an interior portion, and openings extending radially through the outer sleeve from the exterior portion to the interior portion; and a plurality of individually operable actuators located at the exterior portion, each actuator connected to the actuator controller and configured to receive one or more of the coordinated signals, each actuator including a member configured to protract and retract in response to the one or more signals, through one of the openings into and out of the interior portion of the outer sleeve to exert a predetermined force on the body limb and indirectly on at least one of the fragments encircled by the outer sleeve. The apparatus may further include an inner sleeve located in the interior portion of the outer sleeve and dimensioned to encircle the body limb, an interior of the inner sleeve to contact the body limb and an exterior of the inner sleeve to contact the member such that protraction of the member through one of the openings deforms the inner sleeve. The inner sleeve may include a curable cast material.

This invention also provides a bone-repositioning system including an imaging device; a display coupled to the imaging device configured to display an image of a body limb, including fragments of a fractured bone within the body limb, captured by the imaging device; a computing unit coupled to the display and the imaging device, the computing unit configured to receive data from the imaging device, calculate current positions of the fragments based on the data, and determine movement commands to transmit to an actuator controller; an actuator controller coupled to the computing unit, the actuator controller configured to receive the movement commands, translate the commands into a series of coordinated signals, and transmit each signal in the series, wherein each signal is specific to a certain actuator; a rigid outer sleeve dimensioned to encircle the body limb, including the fragments, the outer sleeve having an exterior portion, an interior portion, and openings extending radially through the outer sleeve from the exterior portion to the interior portion; and a plurality of individually operable actuators located at the exterior portion, each actuator connected to the actuator controller and configured to receive the signal specific to the actuator, each actuator including a member configured to protract and retract in response to the one or more signals, through one of the openings into and out of the interior portion of the outer sleeve to exert a predetermined force on the body limb and indirectly on at least one of the fragments encircled by the outer sleeve.

This invention further provides a computer program product including a computer usable medium having computer usable program code for repositioning a fractured bone, the computer program product including computer usable program code for receiving data from an imaging device configured to capture an image of a body limb, including fragments of a fractured bone within the body limb; computer usable program code for calculating a current position of the fragments based on the data; computer usable program code for determining actuator movement commands; and computer usable program code for transmitting the actuator movement commands to a bone-repositioning apparatus coupled to a computing unit executing the computer program product, the bone-repositioning apparatus including an actuator controller configured to receive the actuator movement commands, translate the commands into a series of coordinated signals, and transmit each signal in the series, wherein each signal is specific to a certain actuator; a rigid outer sleeve dimensioned to encircle the body limb, including the fragments, the outer sleeve having an exterior portion, an interior portion, and openings extending radially through the outer sleeve from the exterior portion to the interior portion; and a plurality of individually operable actuators located at the exterior portion, each actuator connected to the actuator controller and configured to receive the signal specific to the actuator, each actuator including a member configured to protract and retract in response to the one or more signals, through one of the openings into and out of the interior portion of the outer sleeve to exert a predetermined force on the body limb and indirectly on at least one of the fragments encircled by the outer sleeve.

These and other features, aspects and advantages of the present invention will become better understood with reference to the following drawings, description and claims.

### BRIEF DESCRIPTION OF THE DRAWINGS

The invention is further described by way of example with reference to the accompanying drawings wherein:

FIG. 1 is a perspective view of an outer sleeve of a bone-repositioning apparatus in accordance with one aspect of this invention.

FIG. 2 is a perspective view of an example body limb having a fractured bone.

FIG. 3A is a perspective view of an outer sleeve and actuators of a bone-repositioning apparatus in accordance with one aspect of this invention.

FIG. 3B is a perspective view of a cut-through of FIG. 3A.

FIGS. 4A-D show side cross-sections of various types of actuator members.

FIG. 5 is a perspective view of an outer sleeve, actuators, and an actuator controller of a bone-repositioning apparatus in accordance with one aspect of this invention.

FIG. 6 is a perspective view of a cut-through of a bone-repositioning apparatus having an inner sleeve in accordance with one aspect of this invention.

FIG. 7 is a cross-sectional view of a bone-repositioning apparatus in accordance with the present invention while in use.

FIG. 8 is a diagram of a bone-repositioning system in accordance with the present invention.

FIG. 9A is a perspective view of an outer sleeve having a hinge in accordance with one aspect of this invention.

FIG. 9B is the outer sleeve of FIG. 9A in an open position.

FIG. 10 is a diagram of a method executed by a bone-repositioning system in accordance with the present invention.

### DETAILED DESCRIPTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention provides an apparatus, system, and process for bone repositioning. A limb with a broken bone inside is enclosed by a sleeve that bears a number of individually driven actuators. The actuators are connected to a computer system which allows a user (e.g., a doctor) to control them individually or in groups. The actuators create defined pressure onto a displaced bone fragment to move it into a desired position. In a preferred embodiment, the doctor has a real-time view of the bone position (e.g. by means of a magnetic resonance imaging (MRI) device, X-ray device, ultrasound device or other visualization device and method) such that he or she can monitor and control the repositioning effectuated by the controlled actuators until the desired bone position has been achieved. In another preferred embodiment the computer system comprises an image processing software that analyzes the picture captured by the MRI, X-ray, ultrasound or other visualization method and calculates there from the actuator settings to effectuate the desired bone position. The software might be fed with other input to make this calculation more precise like medical data of the patient, or even medical data derived from other patients. The system may be used by individuals, e.g. nurses, who would not typically be allowed to perform a manual repositioning.

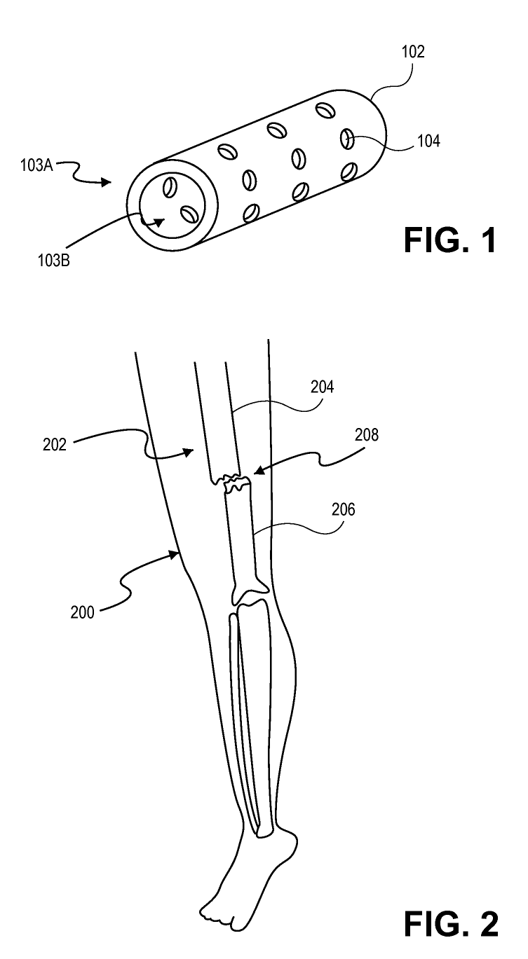

FIG. 1 is a perspective view of an outer sleeve 102 of a bone-repositioning apparatus in accordance with one aspect of this invention. In FIG. 1, the outer sleeve 102 has an exterior portion 103A, an interior portion 103B, and openings 104 extending radially through the outer sleeve 102 from the exterior portion 103A to the interior portion 103B. In FIG. 1, the openings 104 are arranged in an array-like fashion throughout the surface of the outer sleeve 102.

The outer sleeve is rigid and dimensioned to encircle a body limb, including fragments of a fractured bone within the body limb. The body limb could be, for example, the body limb shown in FIG. 2. FIG. 2 is a perspective view of a body limb having a fractured bone. The body limb is a thigh 200. The bone 202 is a femur bone. The bone 202 is fractured at fracture 208 into two fragments: a cranial fragment 204 and a caudal fragment 206. Accordingly, in one use for example, the outer sleeve 102 is dimensioned to encircle the thigh shown in FIG. 2, including both the cranial fragment 204 and the caudal fragment 206 of the broken femur bone 202. In that use, a patient may see the outer sleeve as enveloping around the thigh having the broken femur, the thigh being located generally within the interior portion 103B of the outer sleeve 102.

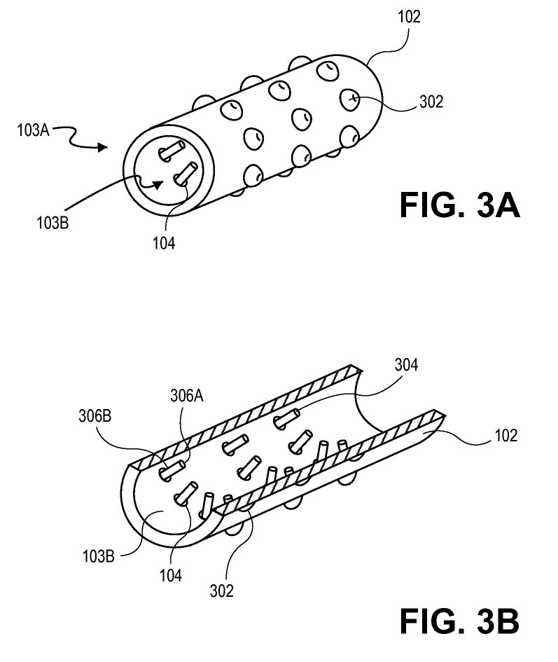

FIG. 3A is a perspective view of an outer sleeve and actuators of a bone-repositioning apparatus in accordance with one aspect of this invention. The actuators 302 are located at the exterior portion 103A of the outer sleeve 102. Each actuator 302 includes a member configured to protract and retract through one of the openings 104 into and out of the interior portion 103B of the outer sleeve 102. Accordingly, in FIG. 3A, the actuators 302 also are arranged in an array-like fashion.

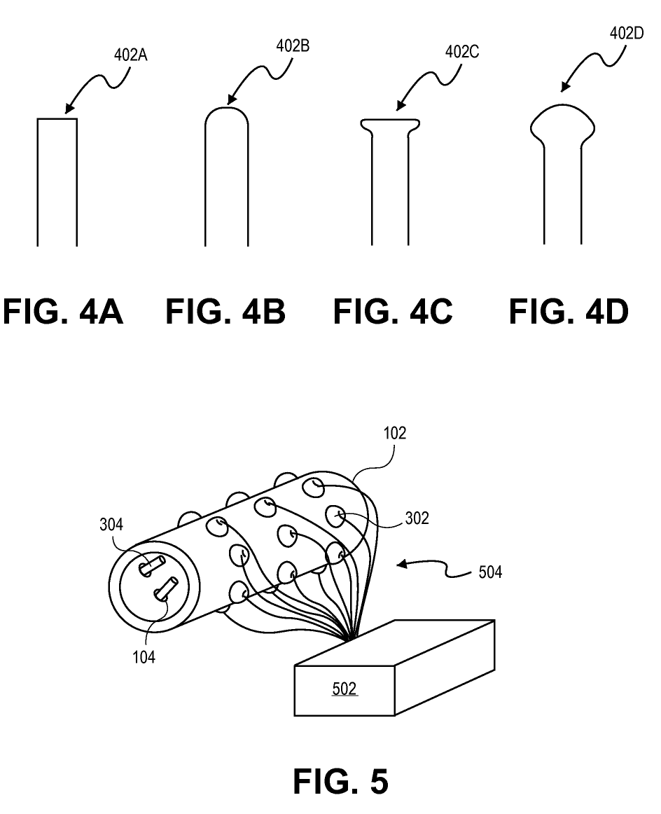

FIG. 3B is a perspective view of a cut-through of FIG. 3A. The member 304 of each of the actuators 302 can be seen in FIG. 3B protruding through the corresponding openings 104 into the interior portion 103B of the outer sleeve 102. The member 304 has a first end 306A proximal to the interior portion and a second end 306B distal to the interior portion. In FIG. 3B, each member 304 has a generally cylindrical shape with a flat surface at the first end 306A. An actuator member in accordance with this invention may have other shapes as well. As examples, FIGS. 4A-D show side cross-sections of the first end of various types of actuator members.

FIG. 4A shows a side cross-section of a member of FIG. 3B. As seen in FIG. 4A, the surface 402A of the first end of the member is generally flat.

FIG. 4B shows a side cross-section of a member having a different shape. In FIG. 4B, the surface 402B of the first end of the member is generally rounded. Accordingly, a bone-repositioning apparatus of the present invention may have a member that has a first end 306A proximal to the interior portion 103B and a second end 306B distal to the interior portion 103B where the first end is rounded.

FIG. 4C shows a side cross-section of a member having yet another shape. In FIG. 4C, similar to FIG. 4A, the surface 402C of the first end of the member is generally flat. However, the member of FIG. 4C differs from the member of FIG. 4A in at least that the member of FIG. 4C has a broader section at its head, i.e. first end 306A. Accordingly, a bone-repositioning apparatus of the present invention may have a member that has a first end 306A proximal to the interior portion 103B and a second end 306B distal to the interior portion 103B where the first end has a larger radius than the second end.

FIG. 4D shows a side cross-section of a member having yet another shape. In FIG. 4D, similar to FIG. 4B, the surface 402C of the first end of the member is rounded. Additionally, in FIG. 4D, similar to FIG. 4C, the first end has a larger radius than the second end. The shape of the member, particular at the first end, effectuates a different pressure distribution against a body limb located in the interior portion of the outer sleeve, as described in more detail below. Depending on the application of the bone-repositioning apparatus, a certain shape may be more beneficial. As such, the shape of the member is a matter of design and can be adjusted to achieve the pressure distribution desired.

In the present invention, each member is individually operable to protract and retract into and out of the interior portion of the outer sleeve. Therefore, although in FIG. 3B, the members 304 appear to be protruding approximately the same distance into the interior portion 103B relative to each other, in use, the distance each member protrudes into the interior portion relative to other members may differ and change. Each member is configured to protract and retract in response to one or more coordinated signals received from an actuator controller. In one application, each member is configured to protract and retract a certain distance based on one or more coordinated signals received from an actuator controller. For example, in use, the actuator controller transmits a signal to an actuator to drive that actuator's member a certain distance into the interior portion of the outer sleeve. Each member may also be configured to protract and retract at a certain speed, in addition, based on one or more coordinated signals received from an actuator controller. For example, in use, the actuator controller may transmit a signal to an actuator to drive that actuator's member both a certain distance and a certain speed into the interior portion of the outer sleeve. The speed may depend on certain parameters, such as the presence of blood vessels or organs close to the fracture, an increase in a measured stress parameter such as heart rate or muscle tension, a decrease in blood pressure, detection of bleeding, a moment when the bone fragments touch each other, and/or detection of patient pain.

FIG. 5 is a perspective view of an outer sleeve, actuators, and an actuator controller of a bone-repositioning apparatus in accordance with one aspect of this invention. As shown in FIG. 5, each actuator 302 is connected to an actuator controller 502. There may be an individual actuator controller 502 for each actuator 302, or as depicted here, a common actuator controller 502 serving a group of or even all actuators 302. Each actuator 302 is configured to receive one or more of the coordinated signals, e.g., via connection devices (e.g., receivers or transceivers) and electrical connections (e.g., wires 504, or also via a wireless connection) or other communications connections connecting the actuator 302 to the actuator controller 502. Each member is configured to protract and retract in response to a signal received from the actuator controller 502. The member may be configured to protract and retract by having or being connected to an electrical motor, for example, which is controllable by the signals. The member may also be configured to protract and retract by being formed of a telescopic rod, for example.

As an example, in use, the outer sleeve 102 encircles the thigh 200 of a patient, including fragments 204 and 206 of the fractured bone 202. The actuator controller 502 is configured to transmit a series of coordinated signals, discussed in more detail below, to the plurality of individually operable actuators 302, each of which is connected to the actuator controller 502 and configured to receive one or more of the coordinated signals. Based on a received signal, each actuator protracts or retracts its member a certain distance and at a certain speed. Because each actuator is individually operable, during operation of the apparatus, some actuators may not protractor or retract while others are protracting and still others are retracting. Each protraction or retraction exerts a predetermined force on the body limb, which in turn exerts a force on the bone fragment 204 and/or the bone fragment 206 to reposition the fragments. Together, the various forces exerted by the individually operable actuators, each moving in a coordinated fashion as managed by the actuator controller 502, reposition the fragments of the fractured bone encircled by the outer sleeve. Typically, the protractions and retractions will occur over time, e.g., in a stepwise and continuous fashion, until the fractured bone is repositioned into its appropriate location.

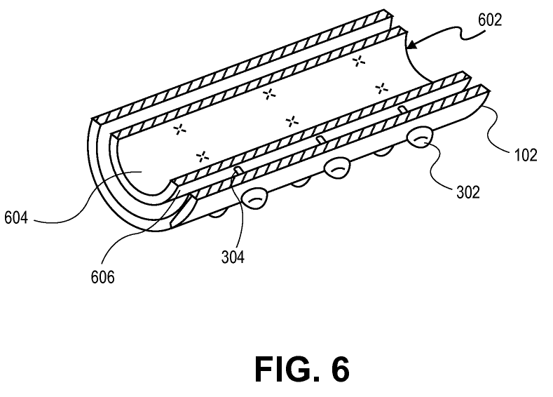

The benefits of a bone-repositioning apparatus in accordance with the present invention may be increased by the use of an inner sleeve. FIG. 6 is a perspective view of a cut-through of a bone-repositioning apparatus having an inner sleeve in accordance with one aspect of this invention. It should be understood that, as a cut-through view, FIG. 6 shows only a part of the apparatus so as to better illustrate certain aspects. In FIG. 6, an inner sleeve 602 is located in the interior portion of the outer sleeve 102. The inner sleeve is dimensioned to encircle a body limb, e.g., the thigh 200. Accordingly, in FIG. 6, while both the inner sleeve and outer sleeve are dimensioned to encircle the body limb, they have different radii. The inner sleeve 602 has a radius that is larger than the radius of the body limb yet smaller than the radius of the outer sleeve 102. The outer sleeve has a radius that is larger than both the radius of the body limb and the radius of the inner sleeve 602.

In FIG. 6, the inner sleeve 602 is made from a material that is deformable, exhibiting elasticity, while the outer sleeve 102 is made from a material that is relatively stiff such that the motion of the members 304 can exert pressure onto the inner sleeve 602 and deform the inner sleeve 602. In FIG. 6, the outer sleeve 102 is made of a material that is sufficiently stiff such that the deformation of the inner sleeve is controllable to a predetermined degree of precision. The inner sleeve 602 is selected to be made of a material that is sufficiently elastic such that pressure on the inner sleeve from a member is distributed in a desirable manner onto the body limb. A material which would distribute too focused a pressure onto the body may not be ideal to effectuate bone repositioning such as a silicon rubber tube or a tissue hose, while a material which would distribute pressure too broadly onto the body limb could have suboptimal effect like a several millimeters thick plexiglass tube or a metallic tube.

In one embodiment, the inner sleeve is composed of a curable cast material such as commonly used fiberglass cast material impregnated with polyurethane, or polymeric materials. The cast can be purely made of such material or also comprise a hose that is filled with the curable material, e.g. if the curable material is not stable enough to form a stable inner sleeve. Also a self-curing material, e.g. a two component mix of a polymerizable methacrylic ester monomeric system comprising a cross-linking methacrylate monomer, some co-monomeric methacrylate diluent and a free radical-generating catalyst, and an accelerator-containing paste system, can be used wherein the preparation of the material is performed in a way that the curing time is synchronized with the repositioning process to occur right after the repositioning has been done. Another possibility is to trigger the curing by the mechanical interaction of the actuators with the material of the inner sleeve. This has the advantage that the curing process is automatically initiated when the repositioning process by means of the actuators is executed.

Having an inner sleeve including a curable cast material can be advantageous at least because, after the bone is repositioned, the inner sleeve can be cured while the limb is held steady by the bone-repositioning apparatus, as described in more detail below. This arrangement reduces the risk of inadvertently altering the position of the fragments that a patient would normally be exposed to in a typical manual, separate and independent process to place an orthopedic cast on the limb. However, alternative methods of replacing the inner sleeve with a cast are also possible and not contrary to this invention.

In one embodiment, the inner sleeve is formed from shell parts that are assembled around the limb. Accordingly, in use, the inner sleeve may be applied in several ways, e.g., by slipping the limb having the fractured bone into the inner sleeve or by assembling the inner sleeve from several parts around the limb. The former method helps ensure a homogenous layer of inner sleeve material is formed around the limb, which in turn helps ensure that predicted effect of members pressing against the inner sleeve are the actual effects. The latter method allows for more customized use of the inner sleeve during treatment. The inner sleeve can be made of a variety of materials, dependent on the functionality requirements for the inner sleeve. If the inner sleeve needs no curability as a property, e.g. if immobilization is provided by other means, it can be made from non-curable materials, and for instance comprise materials that provide flexibility and comfort for the patient, like deformable plastic, leather, rubber, etc. The inner sleeve can also be comprised of a combination of materials, like a layer of fabric material that is arranged inside of a layer of a more rigid material, like the ones mentioned above. A preferred combination is an exterior shell made from a curable cast-material, with an interior shell made from an absorbent material like, for example, a medical gauze material, that allows absortion of sweat from the limb. Another possible material for the inner sleeve is a plastically deformable material that is deformed by the actuators but not as easily deformable in an everyday environment.

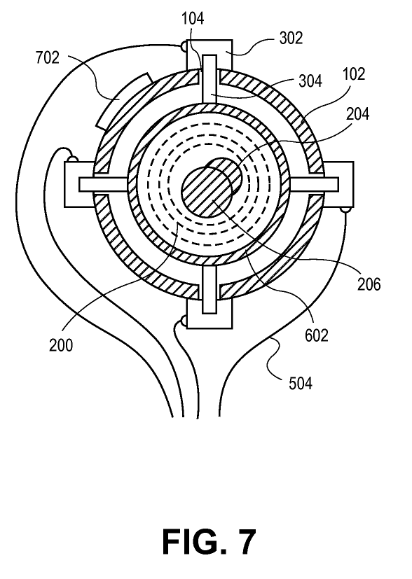

FIG. 7 is a cross-sectional view of a bone-repositioning apparatus in accordance with the present invention while in use. In FIG. 7, the body limb 200 has a bone fracture to be medically treated, namely by repositioning the cranial fragment 204 and the caudal fragment 206 so that they meet again at a desired angle and position. The body limb 200 is positioned to be encircled by the inner sleeve 602 and the outer sleeve 102, e.g., by inserting the body limb into the inner sleeve.

As can be understood by considering both FIG. 6 AND FIG. 7 together, in use, an interior of the inner sleeve, e.g., a surface 604 is in contact with the body limb 200 and an exterior of the inner sleeve, e.g., a surface 606, is in contact with actuator members 304 such that movement of a member 304 through one of the openings 104 of the outer sleeve 102 deforms the inner sleeve 602 in a predetermined area around the point of contact between the member 304 and the inner sleeve 602. The deformation of the inner sleeve 602 exerts a predetermined force on the body limb and indirectly on the fragments 204 and 206 (which are inside the body limb and thus also encircled by the inner sleeve 602 and the outer sleeve 102). The members 304 of the individual actuators 302 move in and out of the outer sleeve 102 in a controlled and coordinated fashion, effectuating the repositioning of the fragments. The repositioning is non-invasive; the members 304 of the actuators do not penetrate the skin of the body limb under treatment. Rather, the members exert forces on the fragments until the fragments are placed in a position within the body limb where they can grow together again in accordance with medically recommended rules of bone growth.

In FIG. 7, an identifier 702 is coupled to the outer sleeve 102. In FIG. 7, the identifier is attached to the exterior portion of the outer sleeve, although the identifier 702 may be coupled to the outer sleeve in other ways, e.g., being embedded within the outer sleeve or attached via a tether. The identifier 702 may be, for example, a radio frequency identification (RFID) tag or a barcode. The identifier 702 may be used in a variety of ways, e.g., to identify the outer sleeve, identify a property of the outer sleeve such as: a type of the outer sleeve (e.g., an outer sleeve for a thigh, for a forearm, or for a lower leg, an outer sleeve for a child or for an adult, or an outer sleeve for individuals in certain weight ranges), a dimension of the outer sleeve (e.g., length, width, radius, depth), or a shape of the member, or any combination of the foregoing. The identifier 702 communicates with a corresponding identifier reader (e.g., an RFID reader or barcode reader) coupled to the actuator controller, as described in more detail below.

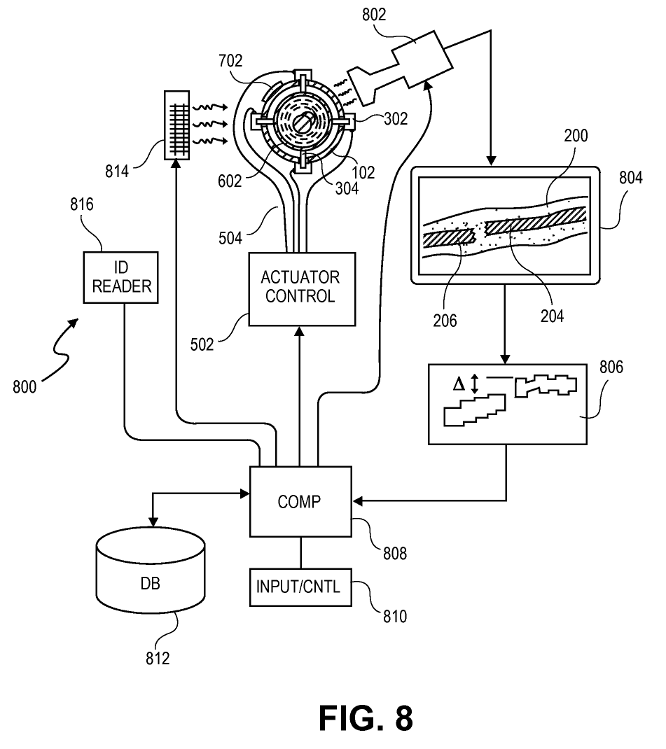

FIG. 8 is a diagram of a bone-repositioning system in accordance with the present invention. In FIG. 8, the components of FIG. 7 are shown within a bone-repositioning system 800. The system includes an imaging device 802, a display 804, a computing unit 808, the actuator controller 502, the outer sleeve 102, and the plurality of individually operable actuators 302. The system further includes an inner sleeve 602, a data sample unit 806, an input/output device 810, a database 812, a curing device 814, and an identifier reader 816. The imaging device 802 may be, for example, an X-ray machine, an ultrasound device, a magnetic resonance imaging device, or any other apparatus that can identify the position of the bone fragments inside a body.

In FIG. 8, the imaging device 802 is coupled to the display 804, the display 804 is coupled to the data sample unit 806, and the data sample unit 806 is coupled to the computing unit 808. The computing unit 808 is further coupled to the actuator controller 502, the input/output device 810, the database 812, the curing device 814, and the identifier reader 816. The actuator controller 502 is coupled to the actuators 302, which may be directly attached to the outer sleeve 102, and whose members are in contact with the inner sleeve 602.

In use, the display 804 is configured to display an image, e.g., an image captured by the imaging device 802. In FIG. 8, the image is of the body limb 200, including the fragments 204 and 206 of the fractured bone within the body limb 200. Display of this image allows the user, e.g., a doctor, to check the image. In certain treatments, the doctor may interfere with the system if the doctor determines based on the image that the system 800 cannot reposition the bone in a desirable manner.

In the system of FIG. 8, the image captured by the imaging device 802 is communicated to the data sample unit 806. The data sample unit 806 uses the information in the image to calculate the relative positions of the bone fragments 204 and 206 within the body limb 200. The data sample unit 806 may use a variety of techniques to calculate the relative positions, e.g. image recognition and/or algorithms described in “Interactive Repositioning of bone fracture segments” by Scheuering et al. (2001). In the system of FIG. 8, the output of the data sample unit 806 is communicated to the computing unit 808.

In some embodiments, the data sample unit 806 is part of the computing unit 808. The computing unit 808 is configured to receive the data from the imaging device 802 and to calculate the current positions of the fragments 204 and 206 based on the data. The computing unit 808 is configured to use the current positions of the fragments (whether or not as an output from the data sample unit 806) to calculate movement commands for the individual actuators 302, and their corresponding members 304. The movement commands are transmitted to the actuator controller 502.

The actuator controller 502 is configured to receive the movement commands determined by the computing unit 808. The actuator controller 502 translates the commands into a series of coordinated signals and transmits each signal in the series to a certain actuator. Each signal effectuates actuator motion. The signal may include data (e.g., a desired distance, speed, and/or direction to move an actuator member) or the signal may simply be an electrical current of a certain magnitude which activates a motor on an actuator a certain amount. Accordingly, each signal is specific to a certain actuator in order to effectuate a specific individual motion, and each actuator 302 is configured to receive the signal specific to that actuator. The actuator is configured to protract and retract (e.g., a certain distance and at a certain speed) in response to the signal it receives. The actuator moves in accordance with that signal to exert a predetermined force on the body limb and indirectly on at least one of the fragments. Together, all the individual motions of the members of the plurality of actuators effectuate an overall repositioning of the bone fragments.

Because the combined motions of all the actuator members determine the effect on the body limb, actuator dependencies can be programmed into the program that calculates the actuator motions. The algorithm may be programmed for instance to preserve the volume of the tissue, or some other dimension. For example, when one member is protracted or pushed in, another member on the opposite side of the sleeve can be retracted or moved out to avoid squeezing the tissue.

The actual movement commands, the coordinated signals, and which actuator receives which signal depends on a variety of factors. The factors may include the location of a specific actuator relative to the fragments, the actual fragment position (e.g., received from the data sample unit 806 or calculated by the computing unit 808), the desired fragment position, the physical properties of the fractured bone under treatment, the measurements of the body limb under treatment, the physical properties of the inner sleeve, the physical properties of the outer sleeve, and the actuator response (e.g., the degree to which a control signal actually translates into a certain motion). Accordingly, the computing unit 808 may be configured to receive these parameters for use in calculating the movement commands. In some applications, the database 812 stores the values of some of these parameters, e.g., the desired fragment position data. Several desirable fragment positions may be stored in the database 812 for selection by a user, e.g., a nurse or doctor. Using the actual fragment position and the desired fragment position, the computing unit 808 calculates the movement commands which will reposition the fragments of the fractured bone.

The computing unit 808 may also use other information to calculate the movement commands, e.g., a physical property of the tissue surrounding the fractured bone, statistical data derived from prior treatments, and a health parameter of the patient under treatment. The physical property of the tissue may be, for example, entered by a physician into the computing unit via the input/output device 810 or extracted from the database 812. The statistical data may be, for example, stored and retrieved from the database 812. The health parameter may be, for example, a heart rate or blood pressure either of which may be an indicator for whether an actuator motion creates a stress effect on the body. Other health parameters may be body temperature, muscle tension or the like. Measuring of other health-related parameters can be performed to ensure that the bone repositioning does not effectuate rupture of a blood vessel or damage to an organ, respectively, or to immediately recognize such damage to enable treatment thereof. The health parameter may be transmitted into the system from another device (not shown in FIG. 8) coupled to the computing unit 808.

In the example above, the system 800 is substantially automated. The imaging device 802, the data sample unit 806, and the computing unit 808 interact to determine which bone to treat based substantially on software, e.g., image recognition software. The computing unit 808 then determines what forces to effectuate on the body limb and reposition the bone fragments. In such an automated system, a feedback or assisted feedback loop may be beneficial.

When a feedback loop is used, the computing unit 808 transmits movement commands to the actuators 302 incrementally. The effects of the movement commands on the fragments' positions are analyzed using a feedback loop of the imaging device 802, the data sample unit 804, and back to the computing unit 808. The actuator motion continues based on the feedback until the bone fragments reach the desired position, determined by analyzing data from the feedback loop. Using such a feedback loop, the system can correct for real-time effects of the actuator movements on a particular patient. However, in certain applications, this feedback loop may expose the patient to bone fragment or tissue damage if the system has little or no assistance in determining which corrective action is most likely to succeed. Accordingly, an assisted feedback loop may be beneficial.

For an assisted feedback loop, other data is used to help the system predetermine what effect a certain motion of an actuator will have. Such data may be, for example, the physical properties of the bones, the physical properties of the limb tissue around the bones, the physical properties of the inner sleeve, the physical properties of the outer sleeve and the actuator response. All such information may be stored, for example, in the database 812. The method through which the information is acquired beforehand may vary. For a specific patient, before applying the bone repositioning method, an analysis of the patient's tissue properties may be performed, e.g. by analyzing a tissue sample. If the patient has been treated before, historical data about that patient may be available. Personal data like age, weight, habits, etc. may be manually entered into the system at the time of treatment or be already present as data in the database 812, and that data can be used to calculate a range of tissue properties which can then be used to better anticipate the effect of the actuator motion and hence make the calculation for the actuator motion to be performed more precise. Finally, if the system 800 has been used several times, the system preferably has historical data available that indicates which actuator motions have been most effective for a specific type of bone fracture and hence are recommendable for reuse. Such historical data is very valuable since it represents direct information from real applications. Historical data can stem from the same patient or even from different people who have been treated before, thereby enhancing the amount of information integrated into the calculation step that determines the actuator motion, and thereby increasing the effectiveness of the system at producing the desired result. Data used from other people can be categorized in a manner such that data that pertains to other people with similar physical parameters (like body weight, tissue density, type of fracture, age, etc.) as the current patient is preferably included in the calculation step. That data can complement theoretical data available from scientific research, which can also be stored and used in the database. Additionally, a networked environment for feeding data to the database is preferred since the data in the database may originate from and be updated from various devices, including devices not shown in FIG. 8.

Use of the above information, although optional for the system 800 to function, increases the effectiveness of the actuator motion and the system's likelihood of achieving a desired bone fragment repositioning more quickly. For example, since a particular motion will be more likely to have the desired effect if the assisting information is taken into account when calculating the motion, the assisted feedback loop can be programmed to perform bigger steps of the members, in a more targeted fashion, to arrive at the target bone fragment position faster.

In addition, the data from the bone repositioning process performed on the patient is also stored in the database 812 for later reuse, e.g. for that same patient or for other patients. In this way, the database may be a recording medium that records treatment history, providing data that allows the system 800 to learn from each treatment performed using the system, or even performed using other similar systems via a network.

In some instances, if the system has enough information that it is to be expected that the actuator motion will with sufficient exactness lead to the desired bone fragment positions, the actuator control can even be programmed to steer all the way through to the final bone fragment position. This is the fastest way to reposition the bone fragments, and uses no feedback.

Depending on the application, a user may provide more input and/or exert more control of the system. The input/output device 810 may be used to exert more or less control, including starting the process as a whole, inputting parameters, stopping the process for interference, and selecting data or parameters. For example, a user, e.g., a nurse, doctor, or other medical professional, may tell the system 800 which bone is to be treated, via input/output device 810. Rather than relying on image recognition software to determine which bone is to be treated, a doctor can use the input/output device 810 to specifically identify to the computing unit 808 which bone to treat. The user may use display 804 to assist in determining or identifying which bone to treat.

In yet other applications, the user indirectly specifies to the computing unit 808 which bone to treat via his/her selection of a specific sleeve. As mentioned above, to determine the specifics of the treatment, the system 800 uses parameters such as the physical properties of the outer sleeve and inner sleeve. Patients have different body limb sizes depending on age, weight, type of limb, etc., so it is advantageous for the system to operate with a set of different sleeves from among which a user can select based on the patient (and body limb) being treated. In applications in which the system 800 is configured to operate using different types of sleeves, the selection of a particular sleeve may indicate to the computing unit 808 which bone is being treated, among other things. For example, selection of a sleeve arrangement in which the inner and outer sleeves are dimensioned for fitting around a forearm rather than around a leg would indicate to the computing unit 808 that the system is treating a fractured ulna or radius rather than a fractured femur. Furthermore, the selection of an outer sleeve intended for a child rather than an adult may indicate to the system 800 that the treatment may be effectuated using less force than other treatments.

The identification of which sleeve is being used may be inputted manually into the computing unit 808 via input/output device 810. Alternatively, the identification may be communicated using the identifier 702. In FIG. 8, the identifier 702 is read by identifier reader 816 which may be an RFID reader or barcode reader, for example, coupled to the actuator controller 502. In FIG. 8, the identifier reader 816 is coupled to the actuator controller 502 via its connection to the computing unit 808. Other wired or wireless communication technology may also be used to identify the outer and/or inner sleeve to the computing unit 808. In some applications, information about the sleeve is communicated directly from the identifier 702 to the computing unit or actuator controller (e.g., when the information is stored on the RFID tag). In some applications, the computing unit 808 uses a received ID to retrieve specific or additional information about the sleeve, e.g. from the database 812. Accordingly, the system 800 can be configured to perform automatic bone recognition (e.g., via image analysis such as that supported by Definiens Incorporated), receive input from a user, e.g., a nurse, doctor, or other medical professional, who tells the system which bone is to be treated, and/or use a sleeve which is configured for specific, predetermined bone types or applications.

The system of FIG. 8 also includes a curing device 814 coupled to the computing unit 808. The curing device 814 is configured to cure the inner sleeve, e.g., by heat, infrared light, ultraviolet light, water, electrical power, or a chemical reaction. In use, once the desired bone position has been achieved, the inner sleeve can be cured. The computing unit 808 may initiate the curing by the curing device or may control the curing process. The cured inner sleeve keeps the limb in the position that was determined by the actuators. By curing the inner sleeve while the limb and inner sleeve are held in place by the outer sleeve and actuator members, a cast is formed around the limb and the risk of inadvertent displacement of the bone during the casting process is significantly reduced and perhaps even eliminated. In the case of a self-curing material of the inner sleeve, the computing unit may be programmed to use the curing time of the material as parameter for the repositioning process. For example, the computing unit 808 may be programmed to perform all actuator movements within a time that is below the curing time, or to take the growing stability of the sleeve material into account when moving the actuators.

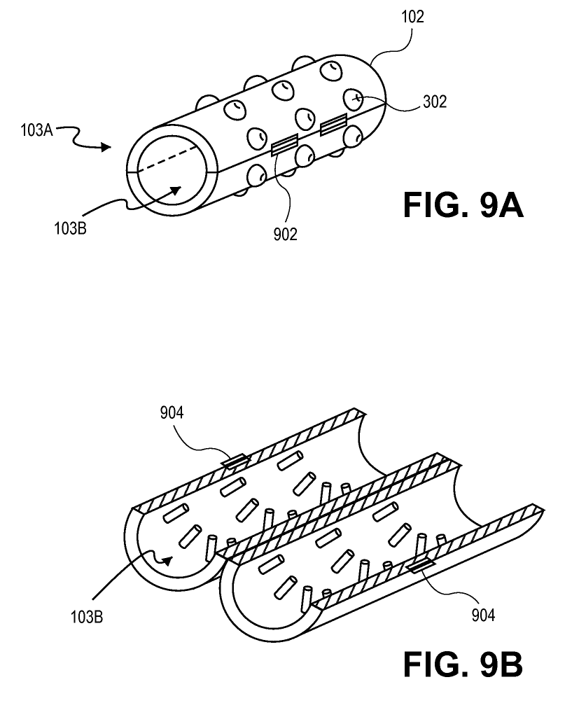

After the inner sleeve is cured, the elasticity has gone and the outer sleeve can be taken off by retracting the members into the actuators and/or opening the outer sleeve. Accordingly, in one embodiment of this invention, the outer sleeve is constructed to be openable and closeable. For example, the outer sleeve may include a hinge configured to convert the outer sleeve from an open position to a closed position. FIG. 9A is a perspective view of an outer sleeve having a hinge in accordance with one aspect of this invention. In FIG. 9A, the outer sleeve is a closed position. FIG. 9B is the outer sleeve of FIG. 9A in an open position. The hinge 902 converts the outer sleeve from the open position to the closed position.

In use, a limb with the inner sleeve around it may be placed within the open outer sleeve. In some embodiments, actuator members 304 are configured to protract into the interior portion 103B of the outer sleeve 102 only when the outer sleeve 102 is in the closed position. The outer sleeve may also have a closing mechanism 904, such as a lock, configured so that the computing unit 808 controls the actuators only when the closing mechanism is engaged and the outer sleeve is closed. In FIG. 9B, the closing mechanism 904 is electronically controllable. In use, the computing unit 808 electronically controls the closing mechanism 904 to prevent opening of the outer sleeve during repositioning.

When the repositioning is complete, the inner sleeve may be cured. In one embodiment, once the curing device has been activated, the computing unit 808 prevents further actuator motion during curing. Once the curing is complete, the outer sleeve is opened. In one embodiment, the computing unit 808 transmits a signal to automatically open the outer sleeve once the curing is complete. Finally the limb with the cast is removed from the outer sleeve. In some applications, the inner sleeve is personalized with a patient id tag for later continuation of the healing treatment process.

In one embodiment, the system is also equipped with a cast splitter that is used during the casting process to provide a gap running longitudinally through the cast, providing the cast with some flexibility to account for postoperative swelling of the limb. The gap may extend radially only through a portion of the cast, or through the entire thickness of the cast. The cast splitter can be implemented as a saw blade arranged within the outer sleeve running along its length and operable by a first saw blade actuator that moves the saw blade along its longitudinal extension. Thereby the saw blade performs a sawing motion. A second saw blade actuator is arranged to move the saw blade radially towards the cast with the limb in order to saw a gap into the cast, thereby splitting it. To avoid damage to the limb, the actuator preferably limits its radial motion to stop before it touches the skin of the limb. Since the system has by means of the imaging device 802 the exact measures of the limb and of the cast splitter, the cast splitting can be conducted with less damage to the limb tissue than would be the case with manual cast splitting.

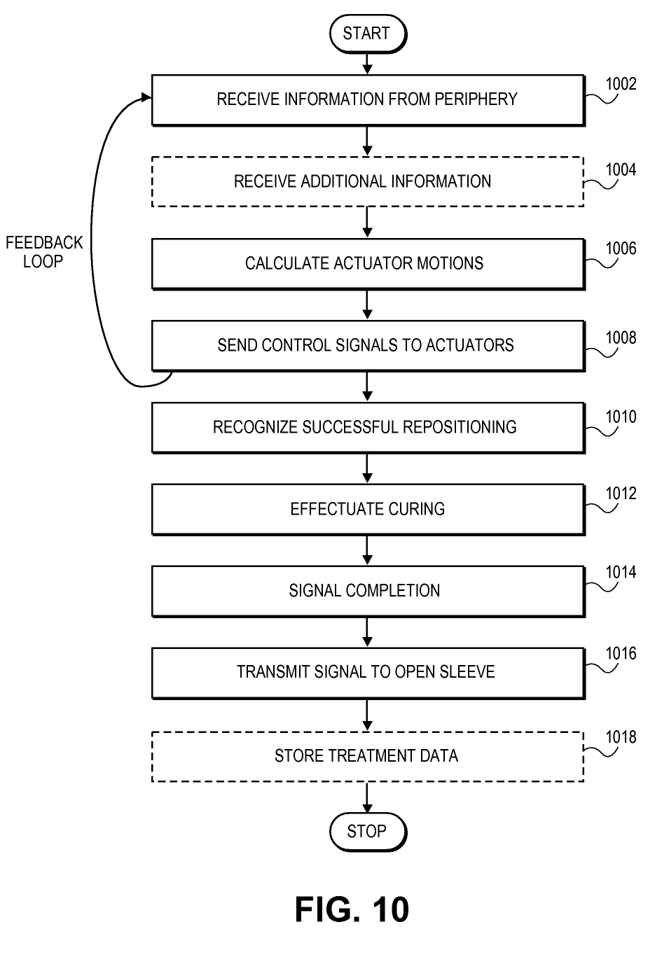

FIG. 10 is a diagram of a method executed by a bone-repositioning system in accordance with the present invention. At 1002, the computing unit 808 receives information from its periphery. For example, in one application, the computing unit 808 includes computer usable program code for receiving data from the imaging device configured to capture an image of a body limb, including fragments of a fractured bone within the body limb. The computing unit may also receive the current bone fragment positions, the bone type and body limb measurements from periphery such as the data sample unit 806 and the input/output device 810. In an embodiment in which the computing unit 808 does not receive a current position of the fragments from the data sample unit 806, the computing unit 808 may include computer usable program code for calculating a current position of the fragments based on the data received from the imaging device. At optional step 1004, the computing unit retrieves additional data from the database 812 such as the various data described above.

At 1006, the computing unit 808 calculates the actuator motions to reposition the bone fragments. For example, in one application, the computing unit 808 includes computer usable program code for determining actuator movement commands.

At 1008, the computing unit sends control signals to the actuators. For example, in one application, the computing unit 808 includes computer usable program code for transmitting the actuator movement commands to the bone-repositioning apparatus described above coupled to the computing unit.

At 1010, the computing unit recognizes successful repositioning via its periphery (e.g., using image analysis). This recognition may be based on feedback response. Accordingly, in one application, the computing unit 808 includes computer usable program code for receiving feedback response from the imaging device 802. Having such feedback response, the computing unit may also include computer usable program code for calculating new actuator movement commands based on the feedback response. In this case the process loops back to 1002, collecting again information such as a new image from the imaging device. This kind of loop can be performed several times until the feedback response signals successful repositioning, e.g. by the image of the repositioned bone fragments being identical or within a predetermined deviation tolerance from the image of a correctly repositioned set of bone fragments. Within the loop the process may also at 1004 collect more additional information from the database, for instance if a complication arises with the bone fragments, e.g. splintering, blocking or the like, wherein the additional information may be retrieved to enhance the repositioning process to cope with the complication. Also, the process may retrieve information selectively in a way that only the information that applies to the planned range of travel distance is retrieved and used. As bone repositioning progresses, information that is relevant for the respective repositioning stage is retrieved as needed.

The process of repositioning the bone fragments can comprise more complex patterns of repositioning movements to be performed, like moving the bone fragments in a circle or another shape of movement path. The computing unit 808 can be programmed to perform such more complex motions by effecting a series of actuator motions.

The system can also be realized as a multi-stage repositioning system, e.g. in the event of a rather extreme limb deformation that does not fit into an inner sleeve that later can become a cast. For this purpose there may be provided a first system that has an outer sleeve only which comprises actuators with a travel distance large enough to provide a coarse repositioning process for the limb. Once the limb has been repositioned closer to its natural position it can be inserted into a second system that now performs the repositioning to the final position with actuators for the fine-positioning.

At 1012, the computing unit effectuates curing of the inner sleeve. For example, in one application, the computing unit 808 includes computer usable program code for transmitting a signal to the curing device 814 to initiate curing of the inner sleeve 602. The signal may initiate curing by heat, infrared light, ultraviolet light, water, electrical power, and/or a chemical reaction.

At 1014, the computing unit outputs a signal that the process is finished and that the limb can be removed from the outer sleeve. At 1016, the computing unit transmits a signal to open the outer sleeve. For example, in one application, the computing unit 808 includes a computer usable program code for transmitting a signal to the closing mechanism 904 to open the outer sleeve 102 when the curing is complete. At 1018, the system may optionally store the treatment data, e.g., in the database 812, for latter use, e.g., by a medical professional in a subsequent treatment of the patient, or in treating other patients.

The system 800 may perform the process of FIG. 10 autonomously once the outer shell has been closed around a body limb. When the system performs the repositioning process autonomously, the system includes, at a minimum, a start/stop input device (e.g., a start/stop button) for safety. In such a system, at “Start” in FIG. 10, the outer sleeve is closed and the start button has been pressed. Thus, in some applications of the above system, supervision by a doctor may not be necessary. Repositioning of a fractured bone may be completed faster, potentially reducing the amount of exposure of the limb to harmful radiation. The exposure is further reduced if for the assisted feedback method the imaging is only performed to generate individual images. One or two images may suffice.

An apparatus, system and method for bone repositioning, particularly computer-aided bone repositioning, is disclosed. In the description above, numerous specific details are set forth in order to provide a thorough understanding of the present invention. However, it will be apparent to one of ordinary skill in the art that these specific details need not be used to practice the present invention. In other circumstances, well-known structures, materials, or processes have not been shown or described in detail in order not to unnecessarily obscure the present invention.

For example, although in FIG. 1 the openings 104 are arranged in an array-like fashion throughout the surface of the outer sleeve 102, in other embodiments, the number of openings and/or the arrangement of those openings may be different than that shown in FIG. 1. For example, the number of openings and/or the arrangement of those openings may be based on factors such as the size of the outer sleeve, the body limb the outer sleeve is designed to encircle, the shape of the outer sleeve, and/or the type of outer sleeve. Consequently, actuators of a bone-repositioning apparatus of the present invention may also be arranged correspondingly. Indeed, the more actuators are arranged the higher is the resolution of deformation points along the inner sleeve and the more precise the repositioning can be performed.

Additionally, although in FIG. 1 the shape of the outer sleeve 102 is cylindrical, in other embodiments, the outer sleeve may be have a different shape, whether generally cylindrical or otherwise. For example, in one embodiment, the outer sleeve has a first end and a second end, and the first end has a greater radius than a second end. Such an outer sleeve may be more appropriate for use in repositioning a broken femur since the cranial part of the thigh is often larger than the caudal part of the thigh. Such an outer sleeve may also be more appropriate for use in repositioning a broken tibia or fibula since the cranial part of the lower leg is often larger than the caudal part of the lower leg.

Furthermore, although in FIG. 8, the system includes a display 804, in other embodiments, e.g., in a bone-repositioning system in accordance with this invention which is used in a fully automated state, the display may be not part of the system.

The described system can be provided as a stationary but also as an entirely mobile system. The components depicted in FIG. 8 can all be integrated into a common housing that is openable to receive the limb to be treated, is thereafter closed around the limb, and then performs the repositioning right there. This is useful if a patient cannot be transported. Bone repositioning and immobilization can thereby be performed at any place, such as right where an accident has happened. Additionally, it is not necessary to integrate all the components of FIG. 8 into a single housing, even for a mobile unit, since using wireless technology, one or more of the components can be located separate and away from the outer sleeve. A preferred embodiment is to provide the outer sleeve with the inner sleeve, the actuators, the actuator control, the imaging device, the curing device, and a power supply within one mobile unit, and configure the remaining components to connect wirelessly to that mobile unit. In particular, the database 812 can be located remotely and connected to via a wireless connection, e.g. using a mobile phone or a satellite connection. In this way, a lightweight repositioning sleeve is provided that is controllable, e.g. by a remote control unit receiving imaging signals from and sending control signals to the repositioning sleeve. In one application, this mobile unit is available as or as part of a first aid kit.

Furthermore, certain configurations of the system described above may be more beneficial to users having limited medical training; other configurations may be more beneficial to users having certain disabilities. Accordingly, the amount of automation, of computer-assistance, may vary depending on the particular application. Additionally, any of the explained methods of selecting a desired fragment position or calculating a repositioning movement may be combined with the other methods described as appropriate for a particular application.

Aspects of the invention can take the form of an entirely hardware embodiment, an entirely software embodiment or an embodiment containing both hardware and software elements. In a preferred embodiment, aspects of the invention are implemented in software, which includes but is not limited to firmware, resident software, microcode, etc.

Furthermore, aspects of the invention can take the form of a computer program product accessible from a computer-usable or computer-readable medium providing program code for use by or in connection with a computer or any instruction execution system. The use of the phase “computer” or the like throughout includes any instruction execution system including but not limited to any computing unit and any data processing system. For the purposes of this description, a computer-usable or computer readable medium can be any apparatus that can contain, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

What is claimed is:

1. A non-invasive bone-repositioning apparatus comprising:
an actuator controller configured to transmit a series of coordinated signals;
a rigid outer sleeve dimensioned to encircle a body limb, including fragments of a fractured bone within the body limb, the outer sleeve having an exterior portion, an interior portion, and openings extending radially through the outer sleeve from the exterior portion to the interior portion; and
a plurality of individually operable actuators located at the exterior portion, each actuator connected to the actuator controller and configured to receive one or more of the coordinated signals, each actuator comprising a member configured to protract and retract in response to the one or more signals, through one of said openings into and out of the interior portion of the outer sleeve to exert a predetermined force on the body limb and indirectly on at least one of the fragments encircled by the outer sleeve.

2. The apparatus of claim 1, further comprising:
an inner sleeve located in the interior portion of the outer sleeve and dimensioned to encircle the body limb, an interior of the inner sleeve to contact the body limb and an exterior of the inner sleeve to contact the member such that protraction of the member through one of said openings deforms the inner sleeve.

3. The apparatus of claim 2, wherein the inner sleeve comprises a curable cast material.

4. The apparatus of claim 1, wherein the member has a first end proximal to the interior portion and a second end distal to the interior portion, and the first end has a larger radius than the second end.

5. The apparatus of claim 1, wherein the member has a first end proximal to the interior portion and a second end distal to the interior portion, and the first end is rounded.

6. The apparatus of claim 1, wherein the outer sleeve has a first end and a second end, and the first end has a greater radius than a second end.

7. The apparatus of claim 1, wherein the outer sleeve includes a hinge configured to convert the outer sleeve from an open position to a closed position.

8. The apparatus of claim 7, wherein the member is configured to protract into the interior portion only when the outer sleeve is in the closed position.

9. A bone-repositioning system comprising:
an imaging device;
a display coupled to the imaging device configured to display an image of a body limb, including fragments of a fractured bone within the body limb, captured by the imaging device;
a computing unit coupled to the display and the imaging device, the computing unit configured to receive data from the imaging device, calculate current positions of the fragments based on the data, and determine movement commands to transmit to an actuator controller;
an actuator controller coupled to the computing unit, the actuator controller configured to receive the movement commands, translate the commands into a series of coordinated signals, and transmit each signal in the series, wherein each signal is specific to a certain actuator;
a rigid outer sleeve dimensioned to encircle the body limb, including the fragments, the outer sleeve having an exterior portion, an interior portion, and openings extending radially through the outer sleeve from the exterior portion to the interior portion; and
a plurality of individually operable actuators located at the exterior portion, each actuator connected to the actuator controller and configured to receive the signal specific to the actuator, each actuator comprising a member configured to protract and retract in response to the one or more signals, through one of said openings into and out of the interior portion of the outer sleeve to exert a predetermined force on the body limb and indirectly on at least one of the fragments encircled by the outer sleeve.

10. The system of claim 9, further comprising:
an inner sleeve located in the interior portion of the outer sleeve and dimensioned to encircle the body limb, an interior of the inner sleeve to contact the body limb and an exterior of the inner sleeve to contact the member such that protraction of the member through one of said openings deforms the inner sleeve.

11. The system of claim 10, wherein the inner sleeve is composed of a curable cast material and the system further comprises:
a curing device coupled to the computing unit, the curing device being configured to cure the inner sleeve by at least one of the following: heat, infrared light, ultraviolet light, water, electrical power, and a chemical reaction.

12. The system of claim 9, further comprising:
an identifier coupled to the outer sleeve, the identifier identifying a property of the outer sleeve selected from the group consisting of: a type of the outer sleeve, a dimension of the outer sleeve, and a shape of the member; and
an identifier reader coupled to the actuator controller.

13. The system of claim 10, wherein the identifier is an RFID tag and the identifier reader is an RFID reader.

14. The system of claim 10, wherein the identifier is a barcode and the identifier reader is a barcode reader.

15. The system of claim 9, wherein the imaging device is selected from the group consisting of an ultrasound device or a magnetic resonance imaging device.

16. The system of claim 9, wherein the computing unit is further configured to receive at least one of the following parameters: a desired fragment position, a physical property of the fractured bone, a physical property of tissue surrounding the fractured bone, a measurement of the body limb, a physical property of the outer sleeve, an actuator response, statistical data derived from a prior treatment, and a health parameter of a patient under treatment.

17. A computer program product comprising a computer usable medium having computer usable program code for repositioning a fractured bone, said computer program product including:
computer usable program code for receiving data from an imaging device configured to capture an image of a body limb, including fragments of a fractured bone within the body limb;
computer usable program code for calculating a current position of the fragments based on the data;
computer usable program code for determining actuator movement commands; and
computer usable program code for transmitting the actuator movement commands to a bone-repositioning apparatus coupled to a computing unit executing the computer program product, the bone-repositioning apparatus comprising:
an actuator controller configured to receive the actuator movement commands, translate the commands into a series of coordinated signals, and transmit each signal in the series, wherein each signal is specific to a certain actuator;
a rigid outer sleeve dimensioned to encircle the body limb, including the fragments, the outer sleeve having an exterior portion, an interior portion, and openings extending radially through the outer sleeve from the exterior portion to the interior portion; and
a plurality of individually operable actuators located at the exterior portion, each actuator connected to the actuator controller and configured to receive the signal specific to the actuator, each actuator comprising a member configured to protract and retract in response to the one or more signals, through one of said openings into and out of the interior portion of the outer sleeve to exert a predetermined force on the body limb and indirectly on at least one of the fragments encircled by the outer sleeve.

18. The computer program product of claim 17, further comprising:
computer usable program code for receiving feedback response from the imaging device; and
computer usable program code for calculating new actuator movement commands based on the feedback response.

19. The computer program product of claim 17, wherein the bone-repositioning apparatus further comprises an inner sleeve comprising a curable cast material, and the computer program product further comprises:
computer usable program code for transmitting a signal to a curing device coupled to the computing unit to initiate curing of the inner sleeve by at least one of the following: heat, infrared light, ultraviolet light, water, electrical power, and a chemical reaction.

20. The computer program product of claim 17, wherein the outer sleeve is configured to open and close, and the computer program product further comprises:
computer usable program code for transmitting a signal to open the outer sleeve when the curing is complete.
